(19) United States
(12) Patent Application Publication
(10) Pub. No.: US 2025/0384962 A1
(43) Pub. Date: Dec. 18, 2025

(54) T-CELL RECEPTOR COMPLEX OPTIMIZATION WITH REINFORCEMENT LEARNING

(71) Applicant: NEC Laboratories America, Inc., Princeton, NJ (US)

(72) Inventors: Tianxiao Li, Plainsboro, NJ (US); Renqiang Min, Princeton, NJ (US); Jonathan Warrell, Princeton, NJ (US)

(21) Appl. No.: 19/235,809

(22) Filed: Jun. 12, 2025

Related U.S. Application Data
(60) Provisional application No. 63/660,791, filed on Jun. 17, 2024.

(51) Int. Cl. G16B 30/20

(52) U.S. Cl. CPC G16B 30/20

(57) ABSTRACT

Systems and methods for particularly t-cell receptor complex optimization with reinforcement learning. Classifiers using variational information bottleneck with attention of experts (AVIB classifiers) can be fine-tuned for different representations of desired t-cell receptor (TCR) sequences for a patient. Proximal policy optimization (PPO) models can be trained with reinforcement learning using the AVIB classifiers as reward functions to achieve higher affinity in generating interaction sequences for the desired TCR sequences through automated decision making. The interaction sequences can be clustered based on k-mer profiles to select the interaction sequences having highest binding scores in each cluster as final sequences. A biological functional potency of the final sequences can be validated.

### RELATED APPLICATION INFORMATION

This application claims priority to U.S. Provisional App. No. 63/660,791, filed on Jun. 17, 2024, incorporated herein by reference in its entirety.

### BACKGROUND

### Technical Field

The present invention relates to specialized t-cell receptor complex optimization using artificial intelligence (AI) models, and more particularly t-cell receptor complex optimization with reinforcement learning.

### Description of the Related Art

Naturally occurring T-cell receptors that exhibit desired properties, such as targeting cancer antigens, are associated with relatively low affinity compared to TCR targeting external pathogens. The proximity of cancer specific sequences to the T-cell receptors can help explain this issue. Engineering an enhanced TCR with modified affinity constitutes a possible solution, however, TCR binding remains challenging to model using structural biology approaches because of the conformational flexibility of the TCR complex. The use of machine learning based methods constitutes a promising approach to design TCR of higher affinity.

### SUMMARY

According to an aspect of the present invention, a computer-implemented method is provided, including, fine-tuning classifiers using variational information bottleneck with attention of experts (AVIB classifiers) for different representations of desired t-cell receptor (TCR) sequences for a patient, training proximal policy optimization (PPO) models with reinforcement learning using the AVIB classifiers as reward functions to achieve higher affinity in generating interaction sequences for the desired TCR sequences, clustering the interaction sequences based on k-mer profiles to select the interaction sequences having highest binding scores in each cluster as final sequences, and validating a biological functional potency of the final sequences.

According to another aspect of the present invention, a system is provided, including, a memory device, one or more processor devices operatively coupled with the memory device to perform operations, fine-tuning classifiers using variational information bottleneck with attention of experts (AVIB classifiers) for different representations of desired t-cell receptor (TCR) sequences for a patient, training proximal policy optimization (PPO) models with reinforcement learning using the AVIB classifiers as reward functions to achieve higher affinity in generating interaction sequences for the desired TCR sequences, clustering the interaction sequences based on k-mer profiles to select the interaction sequences having highest binding scores in each cluster as final sequences, and validating a biological functional potency of the final sequences.

According to yet another aspect of the present invention, a non-transitory computer program product including a computer-readable storage medium having a program code, wherein the program code when executed on a computer causes the computer to perform, fine-tuning classifiers using variational information bottleneck with attention of experts (AVIB classifiers) for different representations of desired t-cell receptor (TCR) sequences for a patient, training proximal policy optimization (PPO) models with reinforcement learning using the AVIB classifiers as reward functions to achieve higher affinity in generating interaction sequences for the desired TCR sequences, clustering the interaction sequences based on k-mer profiles to select the interaction sequences having highest binding scores in each cluster as final sequences, and validating a biological functional potency of the final sequences.

These and other features and advantages will become apparent from the following detailed description of illustrative embodiments thereof, which is to be read in connection with the accompanying drawings.

### BRIEF DESCRIPTION OF DRAWINGS

The disclosure will provide details in the following description of preferred embodiments with reference to the following figures wherein:

FIG. 1 is a block diagram showing a high-level overview of a computer-implemented method for t-cell receptor complex optimization with reinforcement learning, in accordance with an embodiment of the present invention;

FIG. 2 is a block diagram showing a system implementing practical applications of t-cell receptor complex optimization with reinforcement learning, in accordance with an embodiment of the present invention;

FIG. 3 is a block diagram showing a computer system for t-cell receptor complex optimization with reinforcement learning, in accordance with an embodiment of the present invention;

FIG. 4 is a block diagram showing software and hardware components of the computer system for t-cell receptor complex optimization with reinforcement learning, in accordance with an embodiment of the present invention; and

FIG. 5 is a block diagram showing a structure of deep neural networks for t-cell receptor complex optimization with reinforcement learning, in accordance with an embodiment of the present invention.

### DETAILED DESCRIPTION OF PREFERRED EMBODIMENTS

In accordance with embodiments of the present invention, systems and methods are provided for t-cell receptor complex optimization with reinforcement learning.

In an embodiment, classifiers using variational information bottleneck with attention of experts (AVIB classifiers) can be fine-tuned for different representations of desired t-cell receptor (TCR) sequences for a patient. Proximal policy optimization (PPO) models can be trained with reinforcement learning using the AVIB classifiers as reward functions to achieve higher affinity in generating interaction sequences for the desired TCR sequences. The interaction sequences can be clustered based on k-mer profiles to select the interaction sequences having highest binding scores in each cluster as final sequences. A biological functional potency of the final sequences can be validated.

T cells monitor the health status of cells by identifying foreign peptides displayed on their surface. The T-cell receptors (TCRs), protein complexes found on the surface of T cells, can bind to these peptides, which is known as TCR recognition. TCR recognition constitutes a key step for immune response. Optimizing TCR sequences for TCR recognition can be utilized to develop personalized treatments to trigger immune responses to kill cancer cells. However, optimizing TCR sequences for desired properties such as TCR recognition is a difficult task due to the conformational flexibility of the TCR complex which results in an enormous dataset. Consequently, large amounts of computational resources and time is required to optimize TCR sequences.

The present embodiments provide a reinforcement-learning framework based on proximal policy optimization to optimize TCRs through a mutation policy. Briefly after training the system on a series of TCR sequences known to bind a given target, The present embodiments can introduce mutations on existing TCR sequences in order to achieve higher affinity guided by a reward function factoring in affinity of the new sequence and with a high likelihood for such sequence to be valid TCRs.

Due to the mutations introduced through the reinforcement-learning framework, the present embodiments can efficiently optimize TCR sequences for desired properties by compressing the optimization space while retaining maximal information for the desired TCR sequences. Thus, the present embodiments increase the computational cost efficiency of machine learning models for optimizing TCR sequences for desired properties.

The present embodiments generate valid enhanced TCR sequences against the selected epitopes. For example, engineered TCR transfected cells using the present embodiments showed higher activity in the functional assay and demonstrated that TCR generated using the mutation policy can achieve higher biological activity than endogenous TCR. Enhanced TCR generated against MART-1 and KRAS G12V are dissimilar from already described TCR. The engineered TCRs have better antigen recognition compared to their natural state.

Embodiments described herein may be entirely hardware, entirely software or including both hardware and software elements. In a preferred embodiment, the present invention is implemented in software, which includes but is not limited to firmware, resident software, microcode, etc.

Embodiments may include a computer program product accessible from a computer-usable or computer-readable medium providing program code for use by or in connection with a computer or any instruction execution system. A computer-usable or computer readable medium may include any apparatus that stores, communicates, propagates, or transports the program for use by or in connection with the instruction execution system, apparatus, or device. The medium can be magnetic, optical, electronic, electromagnetic, infrared, or semiconductor system (or apparatus or device) or a propagation medium. The medium may include a computer-readable storage medium such as a semiconductor or solid state memory, magnetic tape, a removable computer diskette, a random access memory (RAM), a read-only memory (ROM), a rigid magnetic disk and an optical disk, etc.

Each computer program may be tangibly stored in a machine-readable storage media or device (e.g., program memory or magnetic disk) readable by a general or special purpose programmable computer, for configuring and controlling operation of a computer when the storage media or device is read by the computer to perform the procedures described herein. The inventive system may also be considered to be embodied in a computer-readable storage medium, configured with a computer program, where the storage medium so configured causes a computer to operate in a specific and predefined manner to perform the functions described herein.

A data processing system suitable for storing and/or executing program code may include at least one processor coupled directly or indirectly to memory elements through a system bus. The memory elements can include local memory employed during actual execution of the program code, bulk storage, and cache memories which provide temporary storage of at least some program code to reduce the number of times code is retrieved from bulk storage during execution. Input/output or I/O devices (including but not limited to keyboards, displays, pointing devices, etc.) may be coupled to the system either directly or through intervening I/O controllers.

Network adapters may also be coupled to the system to enable the data processing system to become coupled to other data processing systems or remote printers or storage devices through intervening private or public networks. Modems, cable modem and Ethernet cards are just a few of the currently available types of network adapters.

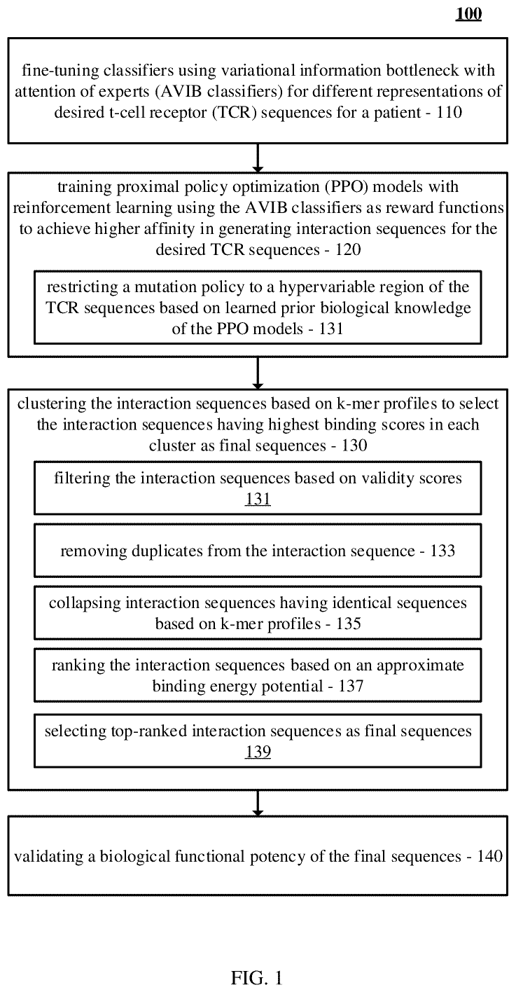

Referring now in detail to the figures in which like numerals represent the same or similar elements and initially to FIG. 1, a block diagram showing a high-level overview of a computer-implemented method for t-cell receptor complex optimization with reinforcement learning, in accordance with an embodiment of the present invention.

In an embodiment, classifiers using variational information bottleneck with attention of experts (AVIB classifiers) can be fine-tuned for different representations of desired t-cell receptor (TCR) sequences for a patient. Proximal policy optimization (PPO) models can be trained with reinforcement learning using the AVIB classifiers as reward functions to achieve higher affinity in generating interaction sequences for the desired TCR sequences. The interaction sequences can be clustered based on k-mer profiles to select the interaction sequences having highest binding scores in each cluster as final sequences. A biological functional potency of the final sequences can be validated

In block 110, fine-tuning classifiers using variational information bottleneck with attention of experts (AVIB classifiers) to determine reward functions for proximal policy optimization (PPO) models for different representations of desired TCR sequences having desired properties for a patient.

A comprehensive fine-tuning dataset for the epitopes of interest can be obtained that combines multiple public datasets. For epitopes with limited records (such as KRAS G12V), positive samples can be curated and mined from literature and randomly shuffled negatives.

AVIB classifiers utilize an attention of experts to perform variational information bottleneck to determine reward functions for proximal policy optimization (PPO) models for different representations of desired TCR sequences having desired properties for a patient. Variational information bottleneck can be used to learn compact, relevant representations of data for classification which can utilize models, such as attention of experts, that can map input data into a latent representation which retains maximal information about a target property while being as compressed as possible from the input.

The attention of experts can include expert models and a gating network. The expert models are neural networks or layers that can be pretrained to perform specific tasks such as classification, for a specific domain such as TCR complexes. The gating network can include a learned component that can decide (e.g., attention over experts) which expert model to utilize for a given input which can output a probability distribution over the expert models having assigned weights. The expert models having the highest weights can be utilized for the given input.

In reinforcement learning, the reward functions can dictate how the PPO models, as the agents, generate their actions based on a state within the environment. In reinforcement learning, agents interact with the environment with actions which are the choices the agent makes within the environment based on a given state. A state can refer to the situation or condition of the environment. And the environment is the external world the agent interacts with.

To determine the reward functions, the AVIB classifiers can be fine-tuned for each epitope to obtain a specialized binding classifier for the epitope which can predict whether an epitope having desired properties can bind with major histocompatibility complex (MHC) molecules. The AVIB classifiers can include a blocks substitution matrix (BLOSUM)-based encoding classifier and a language model-based embedding classifier. The language model-based embedding classifier can include a ProtBERT™ language model-based classifier. The AVIB classifiers can utilize the various classifiers as an ensemble classifier for filtering.

In block 120, training the PPO models with epitopes for the desired TCR sequences with the AVIB classifiers to achieve higher affinity in generating interaction sequences for the desired TCR sequences.

The AVIB classifiers can be utilized as the reward function to train the TCR-PPO policy model. The model learns a mutation policy that maximizes the reward function, resulting in TCR sequences that have high binding score towards the target epitope.

In block 121, a mutation policy can be restricted to a hypervariable region of the TCR sequences based on learned prior biological knowledge of the PPO models.

In an embodiment, the limit on the number of maximal mutations to the hypervariable region of the TCR can be based on a predefined number (e.g., three or five, etc.) learned from prior biological knowledge of the PPO models. This is performed to minimize the modifications on the template. A validity score based on similarity with known TCRs can be incorporated into the reward function.

The trained TCRPPO policy model can perform TCR sequence optimization towards the target epitope through automated decision making. Specifically, to obtain reliable TCRs that can be experimentally validated, the template TCR sequences that interact with the respective MHC complex type can be stratified (e.g., clustered).

In block 130, clustering the interaction sequences based on k-mer profiles to select sequences having the highest binding scores in each cluster as the final sequences.

The interaction sequences can include target epitopes that interact with their respective MHC complex type based on their binding scores. The interaction sequences can be represented with k-mer profiles. The results from iterations of the model (with different classifiers and maximal mutation steps) can be pooled for downstream filtering.

The following processing can be performed on the interaction sequences to obtain the final sequences:

In block 131, the interaction sequences can be filtered based on their validity scores. This filtering process can include removing duplicates, collapsing identical sequences, ranking the sequences, and selecting the top-ranked interaction sequences.

In block 133, duplicates can be removed from the interaction sequences.

In block 135, the interaction sequences having identical sequences can be collapsed. The similarity of the sequences can be determined based on the clustering such as k-mer clustering. In another embodiment, highly similar sequences can be collapsed. For example, similarity can be determined based on the complementarity-determining region 3 (CDR3) sequence similarity, V gene+J gene+CDR3 similarity, unique molecular identifier (UMI) similarity, etc.

In block 137, the interaction sequences can be ranked based on an approximate binding energy potential. In an embodiment, the approximate binding energy can be a Miyazawa-Jernigan potential.

In block 139, the top-ranked interaction sequences can be selected as final sequences.

In block 140, a biological functional potency of the final sequences can be validated.

The final sequences can be validated against known clinically relevant cancer antigens (KRAS G12V and MART-1), or other complexes with desired properties, and evaluated their biological functional potency. To do so, genes encoding variable regions of the original and optimized TCRα and β chains were assembled into plasmid vectors containing a constant region of a TCRα or TCRβ chain. TAP fragments of TCRα and TCRβ together with a NFAT-Luc reporter plasmid were transfected into the ATCR Jurkat cell line. The cells were cultured in the presence of antigen presenting cells with or without target peptide, and then the activation of the reporter gene was measured by luciferase assay.

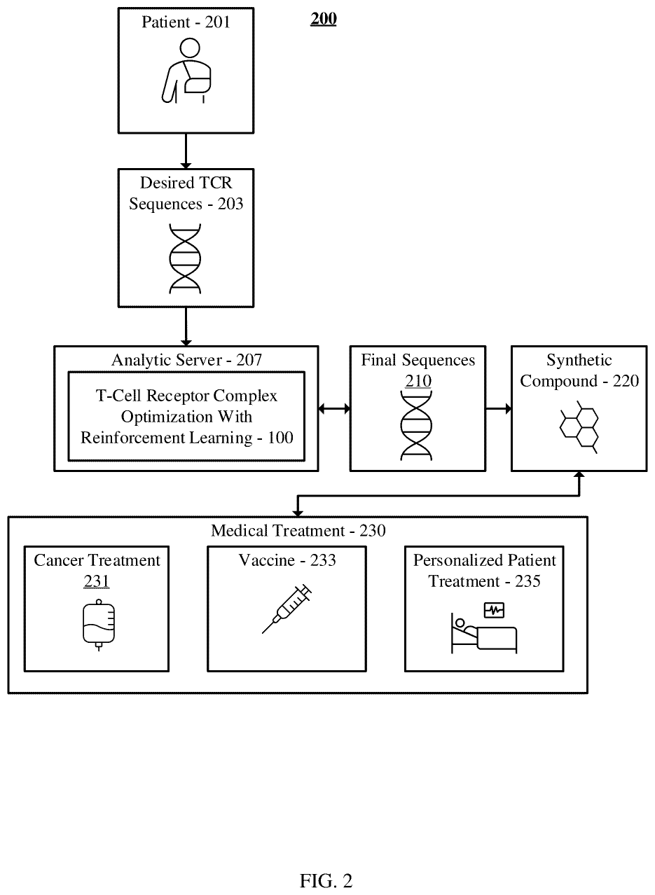

In an embodiment, after validating and manufacturing the final sequences, a medical diagnosis, including a medical treatment based on the medical diagnosis, can be updated based on the final sequences. The medical treatment can be provided to the patient. This is shown in more detail in FIG. 2.

Referring now to FIG. 2, a block diagram showing a system implementing practical applications of t-cell receptor complex optimization with reinforcement learning, in accordance with an embodiment of the present invention.

In system 200, patient 201 can be diagnosed where targeted information about the disease of patient 201 (e.g., cancer, rare genetic diseases, etc.) can be identified having desired TCR sequences 203 through automated decision making. An analytic server 207 can implement t-cell receptor complex optimization with reinforcement learning 100 to identify final sequences 210.

The final sequences 210 can be engineered (e.g., single-cell ribonucleic acid (RNA) sequencing, retrovirus engineering, clustered regularly interspaced short palindromic repeats (CRISPR) targeted genome editing, etc.) to be usable for downstream applications such as cancer treatment 211, vaccine 213, and personalized patient treatment 215. Other engineering processes can be utilized.

To develop cancer treatment 211, the desired TCR sequences 203 can be identified and processed to target cancer cells. The final sequences 210 can be engineered as antibodies that can include T-cells. T-cells can recognize antigens (e.g., MHC-peptide) on abnormal cells (e.g., cancer cells) and can be expressed as TCRs. The TCR can bind to the antigen and the T-cell can release toxic chemicals that can destroy the antigens. The cancer treatment 211 can be provided intravenously, orally, or with other appropriate administration route. Examples of cancer treatment 211 can include chimeric antigen receptor (CAR) T-cell therapy (e.g., Tisagenlecluecel), CAR natural killer (NK) cell therapy, etc.

To develop a vaccine 213, the desired TCR sequences 203 can be identified and processed to target infectious diseases such as influenza, tuberculosis, etc. The final sequences 210 can be engineered as antibodies that can include T-cells. T-cells have TCRs that can recognize pathogens (e.g., having a pMHC) such as viruses, bacteria, fungi, and parasites). The TCR can bind to the antigen and the T-cell can release toxic chemicals that can destroy the pathogens. The vaccine 213 can be provided to the patient 201 intravenously, orally, or with other appropriate administration route. Examples of vaccine 213 can include protein-based vaccines such as conjugate vaccines (e.g., for pneumonia such as pneumococcal conjugate vaccine, meningitis, etc.), recombinant protein vaccines (e.g., for shingles, hepatitis B, etc.), polysaccharide vaccines (e.g., for pneumonia, meningitis, etc.), etc.

To develop personalized patient treatment 215, the desired TCR sequences 203 can be identified and processed to target the illness of patient 201. The final sequences 210 can be engineered as antibodies that can include T-cells. T-cells can recognize antigens (e.g., pMHC) on abnormal cells that causes the illness of patient 201 (e.g., abnormal cells caused by an abnormal mutation in healthy cells) and can be expressed as TCRs. The TCR can bind to the antigen and the T-cell can release toxic chemicals that can destroy the antigens. The personalized patient treatment 215 can be provided to the patient 201 intravenously, orally, or with other appropriate administration route. Examples of personalized patient treatment 215 can include chimeric antigen receptor (CAR) T-cell therapy (e.g., Tisagenlecluecel), gene therapy drug for atopic dermatitis (e.g., abrocitinib), gene therapy drug for hemolytic anema (e.g., mitapivat) etc.

In another embodiment, the system 200 can also perform other downstream tasks such as classification of objects, data anomaly detection, object identification, scene reconstruction, etc.

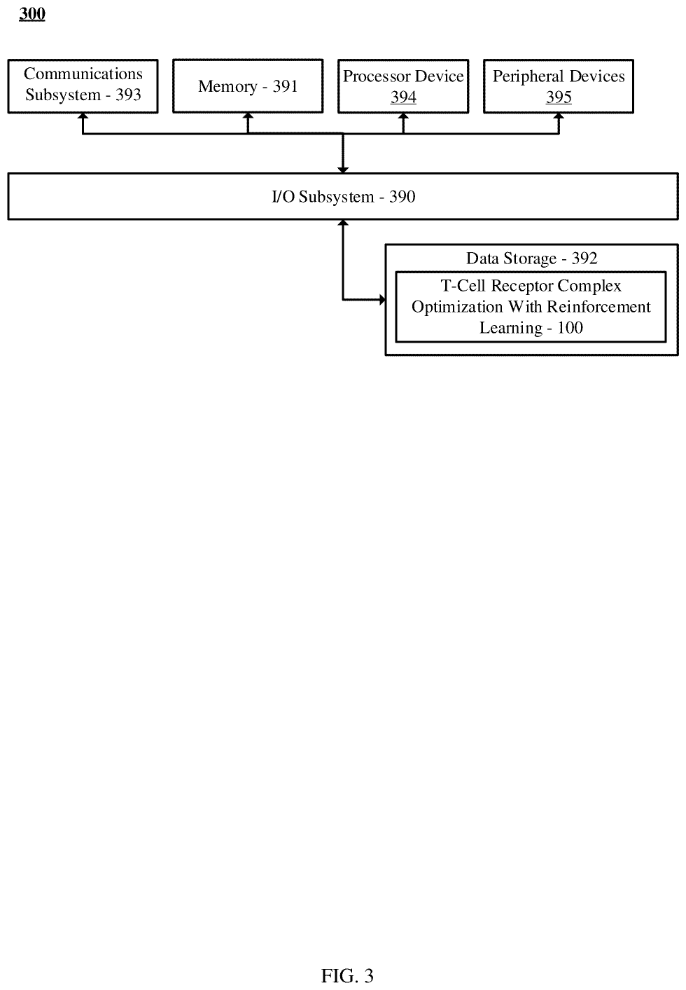

Referring now to FIG. 3, a block diagram showing a computer system for t-cell receptor complex optimization with reinforcement learning, in accordance with an embodiment of the present invention.

The computing device 300 illustratively includes the processor device 394, an input/output (I/O) subsystem 390, a memory 391, a data storage device 392, and a communication subsystem 393, and/or other components and devices commonly found in a server or similar computing device. The computing device 300 may include other or additional components, such as those commonly found in a server computer (e.g., various input/output devices), in other embodiments. Additionally, in some embodiments, one or more of the illustrative components may be incorporated in, or otherwise form a portion of, another component. For example, the memory 391, or portions thereof, may be incorporated in the processor device 394 in some embodiments.

The processor device 394 may be embodied as any type of processor capable of performing the functions described herein. The processor device 394 may be embodied as a single processor, multiple processors, a Central Processing Unit(s) (CPU(s)), a Graphics Processing Unit(s) (GPU(s)), a single or multi-core processor(s), a digital signal processor(s), a microcontroller(s), or other processor(s) or processing/controlling circuit(s).

The memory 391 may be embodied as any type of volatile or non-volatile memory or data storage capable of performing the functions described herein. In operation, the memory 391 may store various data and software employed during operation of the computing device 300, such as operating systems, applications, programs, libraries, and drivers. The memory 391 is communicatively coupled to the processor device 394 via the I/O subsystem 390, which may be embodied as circuitry and/or components to facilitate input/output operations with the processor device 394, the memory 391, and other components of the computing device 300. For example, the I/O subsystem 390 may be embodied as, or otherwise include, memory controller hubs, input/output control hubs, platform controller hubs, integrated control circuitry, firmware devices, communication links (e.g., point-to-point links, bus links, wires, cables, light guides, printed circuit board traces, etc.), and/or other components and subsystems to facilitate the input/output operations. In some embodiments, the I/O subsystem 390 may form a portion of a system-on-a-chip (SOC) and be incorporated, along with the processor device 394, the memory 391, and other components of the computing device 300, on a single integrated circuit chip.

The data storage device 392 may be embodied as any type of device or devices configured for short-term or long-term storage of data such as, for example, memory devices and circuits, memory cards, hard disk drives, solid state drives, or other data storage devices. The data storage device 392 can store program code for t-cell receptor complex optimization with reinforcement learning 100. Any or all of these program code blocks may be included in a given computing system.

The communication subsystem 393 of the computing device 300 may be embodied as any network interface controller or other communication circuit, device, or collection thereof, capable of enabling communications between the computing device 300 and other remote devices over a network. The communication subsystem 393 may be configured to employ any one or more communication technology (e.g., wired or wireless communications) and associated protocols (e.g., Ethernet, InfiniBand®, Bluetooth®, Wi-Fi®, WiMAX, etc.) to effect such communication.

As shown, the computing device 300 may also include one or more peripheral devices 395. The peripheral devices 395 may include any number of additional input/output devices, interface devices, and/or other peripheral devices. For example, in some embodiments, the peripheral devices 395 may include a display, touch screen, graphics circuitry, keyboard, mouse, speaker system, microphone, network interface, and/or other input/output devices, interface devices, GPS, camera, and/or other peripheral devices.

Of course, the computing device 300 may also include other elements (not shown), as readily contemplated by one of skill in the art, as well as omit certain elements. For example, various other sensors, input devices, and/or output devices can be included in computing device 300, depending upon the particular implementation of the same, as readily understood by one of ordinary skill in the art. For example, various types of wireless and/or wired input and/or output devices can be employed. Moreover, additional processors, controllers, memories, and so forth, in various configurations can also be utilized. These and other variations of the computing device 300 are readily contemplated by one of ordinary skill in the art given the teachings of the present invention provided herein.

As employed herein, the term “hardware processor subsystem” or “hardware processor” can refer to a processor, memory, software or combinations thereof that cooperate to perform one or more specific tasks. In useful embodiments, the hardware processor subsystem can include one or more data processing elements (e.g., logic circuits, processing circuits, instruction execution devices, etc.). The one or more data processing elements can be included in a central processing unit, a graphics processing unit, and/or a separate processor- or computing element-based controller (e.g., logic gates, etc.). The hardware processor subsystem can include one or more on-board memories (e.g., caches, dedicated memory arrays, read only memory, etc.). In some embodiments, the hardware processor subsystem can include one or more memories that can be on or off board or that can be dedicated for use by the hardware processor subsystem (e.g., ROM, RAM, basic input/output system (BIOS), etc.).

In some embodiments, the hardware processor subsystem can include and execute one or more software elements. The one or more software elements can include an operating system and/or one or more applications and/or specific code to achieve a specified result.

In other embodiments, the hardware processor subsystem can include dedicated, specialized circuitry that performs one or more electronic processing functions to achieve a specified result. Such circuitry can include one or more application-specific integrated circuits (ASICs), field-programmable gate arrays (FPGAs), and/or programmable logic arrays (PLAs).

These and other variations of a hardware processor subsystem are also contemplated in accordance with embodiments of the present invention.

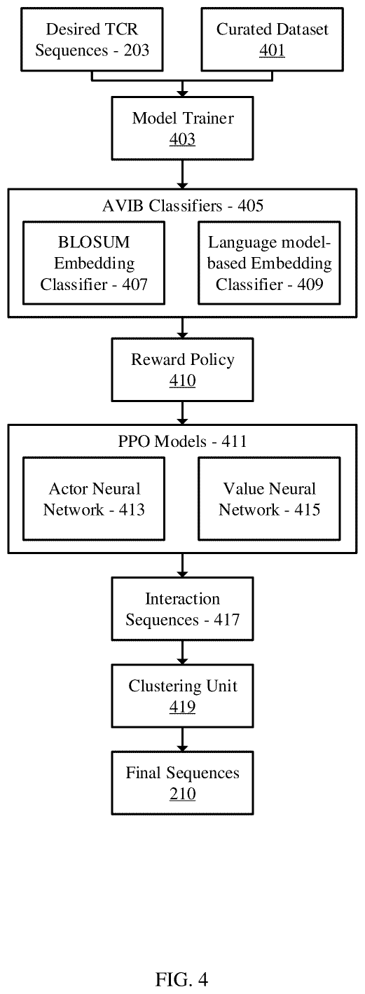

Referring now to FIG. 4, a block diagram showing software and hardware components of the computer system for t-cell receptor complex optimization with reinforcement learning, in accordance with an embodiment of the present invention.

In system 300, desired TCR sequences 203 and a curated dataset 401 can be utilized by a model trainer 403 to train AVIB classifiers 405 and PPO models 411. The AVIB classifiers 405 can include a BLOSUM embedding classifier 407 and a language model-based embedding classifier 409 to determine a reward policy 410. The reward policy 410 can be utilized by the model trainer 403 to train the PPO models 411 to generate interaction sequences 417. The PPO models 411 can include an actor neural network 413 and a value neural network 415. The actor neural network 413 can utilize states of the environment and outputs an action to maximize an expected reward. The value neural network 415 can utilize states of the environment and outputs an expected return.

The interaction sequences 417 can be processed by the clustering unit 419 to obtain final sequences 210 by filtering the interaction sequences 417 based on their k-mer profiles and obtaining the highest-ranked interaction sequences as the final sequences 210.

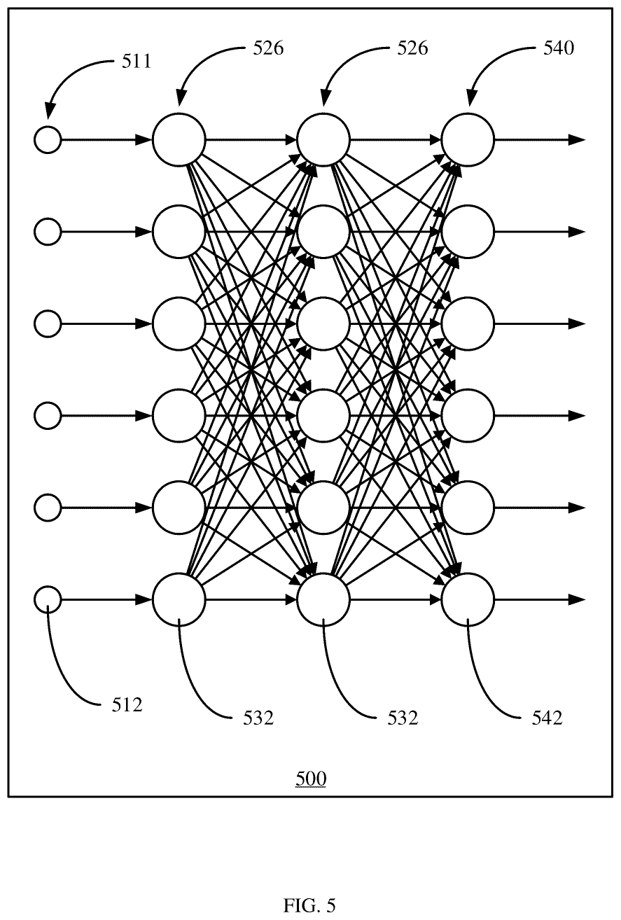

Referring now to FIG. 5, a block diagram showing a structure of deep neural networks for t-cell receptor complex optimization with reinforcement learning, in accordance with an embodiment of the present invention.

A neural network is a generalized system that improves its functioning and accuracy through exposure to additional empirical data. The neural network becomes trained by exposure to the empirical data. During training, the neural network stores and adjusts a plurality of weights that are applied to the incoming empirical data. By applying the adjusted weights to the data, the data can be identified as belonging to a particular predefined class from a set of classes or a probability that the inputted data belongs to each of the classes can be output.

The empirical data, also known as training data, from a set of examples can be formatted as a string of values and fed into the input of the neural network. Each example may be associated with a known result or output. Each example can be represented as a pair, (x, y), where x represents the input data and y represents the known output. The input data may include a variety of different data types and may include multiple distinct values. The network can have one input neurons for each value making up the example's input data, and a separate weight can be applied to each input value. The input data can, for example, be formatted as a vector, an array, or a string depending on the architecture of the neural network being constructed and trained.

The neural network “learns” by comparing the neural network output generated from the input data to the known values of the examples and adjusting the stored weights to minimize the differences between the output values and the known values. The adjustments may be made to the stored weights through back propagation, where the effect of the weights on the output values may be determined by calculating the mathematical gradient and adjusting the weights in a manner that shifts the output towards a minimum difference. This optimization, referred to as a gradient descent approach, is a non-limiting example of how training may be performed. A subset of examples with known values that were not used for training can be used to test and validate the accuracy of the neural network.

During operation, the trained neural network can be used on new data that was not previously used in training or validation through generalization. The adjusted weights of the neural network can be applied to the new data, where the weights estimate a function developed from the training examples. The parameters of the estimated function which are captured by the weights are based on statistical inference.

The deep neural network 500, such as a multilayer perceptron, can have an input layer 511 of source neurons 512, one or more computation layer(s) 526 having one or more computation neurons 532, and an output layer 540, where there is a single output neuron 542 for each possible category into which the input example could be classified. An input layer 511 can have a number of source neurons 512 equal to the number of data values 512 in the input data 511. The computation neurons 532 in the computation layer(s) 526 can also be referred to as hidden layers, because they are between the source neurons 512 and output neuron(s) 542 and are not directly observed. Each neuron 532, 542 in a computation layer generates a linear combination of weighted values from the values output from the neurons in a previous layer, and applies a non-linear activation function that is differentiable over the range of the linear combination. The weights applied to the value from each previous neuron can be denoted, for example, by w₁, W₂, . . . . Wₙ₋₁, Wₙ. The output layer provides the overall response of the network to the inputted data. A deep neural network can be fully connected, where each neuron in a computational layer is connected to all other neurons in the previous layer, or may have other configurations of connections between layers. If links between neurons are missing, the network is referred to as partially connected.

Training a deep neural network can involve two phases, a forward phase where the weights of each neuron are fixed and the input propagates through the network, and a backwards phase where an error value is propagated backwards through the network and weight values are updated. The computation neurons 532 in the one or more computation (hidden) layer(s) 526 perform a nonlinear transformation on the input data 512 that generates a feature space. The classes or categories may be more easily separated in the feature space than in the original data space.

In an embodiment, the computation layers 526 of the AVIB classifiers 405 can learn relationships between a curated dataset 401 and the desired TCR sequences to determine a reward policy 410 that achieve higher affinity for interactions with the desired TCR sequences. The output layer 542 can then generate trained AVIB classifiers 405 that can be utilized as the reward policy 410. In an embodiment, the computation layers 526 of the PPO models 411 can learn relationships between the states of the environment including the desired TCR sequences 203 and the curated dataset 401 to determine the interaction sequences 417. The output layer 542 of the PPO models 411 can then generate a prediction of the interaction sequences 417.

Reference in the specification to “one embodiment” or “an embodiment” of the present invention, as well as other variations thereof, means that a particular feature, structure, characteristic, and so forth described in connection with the embodiment is included in at least one embodiment of the present invention. Thus, the appearances of the phrase “in one embodiment” or “in an embodiment”, as well any other variations, appearing in various places throughout the specification are not necessarily all referring to the same embodiment. However, it is to be appreciated that features of one or more embodiments can be combined given the teachings of the present invention provided herein.

It is to be appreciated that the use of any of the following “/”, “and/or”, and “at least one of”, for example, in the cases of “A/B”, “A and/or B” and “at least one of A and B”, is intended to encompass the selection of the first listed option (A) only, or the selection of the second listed option (B) only, or the selection of both options (A and B). As a further example, in the cases of “A, B, and/or C” and “at least one of A, B, and C”, such phrasing is intended to encompass the selection of the first listed option (A) only, or the selection of the second listed option (B) only, or the selection of the third listed option (C) only, or the selection of the first and the second listed options (A and B) only, or the selection of the first and third listed options (A and C) only, or the selection of the second and third listed options (B and C) only, or the selection of all three options (A and B and C). This may be extended for as many items listed.

The foregoing is to be understood as being in every respect illustrative and exemplary, but not restrictive, and the scope of the invention disclosed herein is not to be determined from the Detailed Description, but rather from the claims as interpreted according to the full breadth permitted by the patent laws. It is to be understood that the embodiments shown and described herein are only illustrative of the present invention and that those skilled in the art may implement various modifications without departing from the scope and spirit of the invention. Those skilled in the art could implement various other feature combinations without departing from the scope and spirit of the invention. Having thus described aspects of the invention, with the details and particularity required by the patent laws, what is claimed and desired protected by Letters Patent is set forth in the appended claims.

What is claimed is:

1. A computer-implemented method, comprising:
fine-tuning classifiers using variational information bottleneck with attention of experts (AVIB classifiers) for different representations of desired t-cell receptor (TCR) sequences for a patient;
training proximal policy optimization (PPO) models with reinforcement learning using the AVIB classifiers as reward functions to achieve higher affinity in generating interaction sequences for the desired TCR sequences;
clustering the interaction sequences based on k-mer profiles to select the interaction sequences having highest binding scores in each cluster as final sequences; and
validating a biological functional potency of the final sequences.

2. The computer-implemented method of claim 1, wherein training the PPO models further comprises restricting a mutation policy to a hypervariable region of the TCR sequences based on learned prior biological knowledge of the PPO models.

3. The computer-implemented method of claim 1, wherein clustering the interaction sequences further comprises filtering the interaction sequences based on validity scores.

4. The computer-implemented method of claim 2, wherein clustering the interaction sequences further comprises removing duplicates from the interaction sequences.

5. The computer-implemented method of claim 2, wherein clustering the interaction sequences further comprises collapsing interaction sequences having identical sequences based on k-mer profiles.

6. The computer-implemented method of claim 2, wherein clustering the interaction sequences further comprises ranking the interaction sequences based on an approximate binding energy potential.

7. The computer-implemented method of claim 2, wherein clustering the interaction sequences further comprises selecting top-ranked interaction sequences as final sequences.

8. A system, comprising:
a memory device;
one or more processor devices operatively coupled with the memory device to perform operations:
fine-tuning classifiers using variational information bottleneck with attention of experts (AVIB classifiers) for different representations of desired t-cell receptor (TCR) sequences for a patient;
training proximal policy optimization (PPO) models with reinforcement learning using the AVIB classifiers as reward functions to achieve higher affinity in generating interaction sequences for the desired TCR sequences;
clustering the interaction sequences based on k-mer profiles to select the interaction sequences having highest binding scores in each cluster as final sequences; and
validating a biological functional potency of the final sequences.

9. The system of claim 8, wherein training the PPO models further comprises restricting a mutation policy to a hypervariable region of the TCR sequences based on learned prior biological knowledge of the PPO models.

10. The system of claim 9, wherein clustering the interaction sequences further comprises filtering the interaction sequences based on validity scores.

11. The system of claim 9, wherein clustering the interaction sequences further comprises removing duplicates from the interaction sequences.

12. The system of claim 9, wherein clustering the interaction sequences further comprises collapsing interaction sequences having identical sequences based on k-mer profiles.

13. The system of claim 9, wherein clustering the interaction sequences further comprises ranking the interaction sequences based on an approximate binding energy potential.

14. The system of claim 9, wherein clustering the interaction sequences further comprises selecting top-ranked interaction sequences as final sequences.

15. A non-transitory computer program product comprising a computer-readable storage medium including a program code, wherein the program code when executed on a computer causes the computer to perform:
fine-tuning classifiers using variational information bottleneck with attention of experts (AVIB classifiers) for different representations of desired t-cell receptor (TCR) sequences for a patient;
training proximal policy optimization (PPO) models with reinforcement learning using the AVIB classifiers as reward functions to achieve higher affinity in generating interaction sequences for the desired TCR sequences;
clustering the interaction sequences based on k-mer profiles to select the interaction sequences having highest binding scores in each cluster as final sequences; and
validating a biological functional potency of the final sequences.

16. The non-transitory computer program product of claim 15, wherein training the PPO models further comprises restricting a mutation policy to a hypervariable region of the TCR sequences based on learned prior biological knowledge of the PPO models.

17. The non-transitory computer program product of claim 16, wherein clustering the interaction sequences further comprises filtering the interaction sequences based on validity scores.

18. The non-transitory computer program product of claim 16, wherein clustering the interaction sequences further comprises removing duplicates from the interaction sequences.

19. The non-transitory computer program product of claim 16, wherein clustering the interaction sequences further comprises collapsing interaction sequences having identical sequences based on k-mer profiles.

20. The non-transitory computer program product of claim 16, wherein clustering the interaction sequences further comprises selecting top-ranked interaction sequences based on an approximate binding energy potential as final sequences.
